(19) United States
(12) Patent Application Publication
(10) Pub. No.: US 2018/0223103 A1
(43) Pub. Date: Aug. 9, 2018

(54) COLOURING OF SURFACES

(71) Applicant: Merck Patent GmbH, Darmstadt (DE)

(72) Inventor: Ralf SCHWEINFURTH, Darmstadt (DE)

(73) Assignee: Merck Patent GmbH, Darmstadt (DE)

(21) Appl. No.: 15/890,770

(22) Filed: Feb. 7, 2018

(30) Foreign Application Priority Data
Feb. 7, 2017 (DE) 10 2017 001 107.9

(51) Int. Cl. C09C 1/00; A23P 20/10; A23L 5/43

(52) U.S. Cl. CPC C09C 1/0051; A23P 20/10; A23L 5/43; C09C 2200/1004; C09C 2200/304; C09C 2200/102; C09C 2200/308

(57) ABSTRACT

The present invention relates to a process for colouring surfaces, in particular surfaces of foods, with effect pigments, preferably pearlescent pigments, based on flake-form substrates, where (i) the effect pigment is incorporated into a pasty, transparent, water-based material and (ii) the application medium prepared in step (i) is applied to the surface.

The present invention relates to a process for colouring surfaces, in particular surfaces of foods, with effect pigments based on flake-form substrates.

Other than for functional uses, effect pigments, such as, for example, metal, pearlescent and interference pigments, are increasingly being employed for enhancing the appearance of products, for example in cosmetics, since attractive colours and effects bring about pleasant subjective impressions for the observer and consumer. In the preparation of pearlescent pigments, for example for the foods sector, stringent requirements are made of the purity and quality of the pigments. Pearlescent pigments are already being employed in the foods sector for improving the colour effect or for imparting colour.

Pearlescent pigments are applied to surfaces, for example surfaces of foods, or incorporated into foods in a very wide variety of ways. In the case of application to a surface, for example a food surface, any application medium used for the application must be selected to match the food, depending on the desired colour effect and type of food. The term application medium here is taken generally taken to mean a fluid into which the pearlescent pigment is incorporated for the purpose of application. Any application method used also has a significant influence on the quality and quantity of the pearlescent effect. The desired application method is frequently not suitable for the food, or is not suitable or of only limited suitability for the application medium that is possible or desired for the colouring.

Pearlescent pigments are usually applied to surfaces by, for example, spraying methods and optional subsequent drying steps. This procedure is complex and expensive. On use of chocolate-flavoured coatings as application medium for pearlescent pigments, the cleaning effort as a consequence of the process may make a considerable contribution to the costs and process times. If pearlescent pigments are used directly, i.e. without the use of an application medium, there is a risk of cross-contamination by pigment dusts in the production area.

The object of the present invention is therefore to provide a simple process for the application of pearlescent pigments to surfaces which in addition can be applied to a multiplicity of surfaces, in particular surfaces of a wide very wide variety of foods, and at the same time broadens the range of existing application methods with regard to cost effectiveness, visual impression of the surface and practicability.

Surprisingly, it has been found that a pearlescent pigment can advantageously be applied to a surface by a process in which the pearlescent pigment is firstly incorporated into a pasty, transparent, water-based application medium and this mixture is subsequently applied to the surface.

A pasty medium can be, for example, a food product showing a medium or high viscosity compared to low viscosity fluids like, i.e., water, where the viscosity can be determined by SI unit: Pa·s.

Preferred effect pigments are pearlescent pigments and interference pigments based on flake-form substrates which have been coated with one or more metal oxides and/or metal hydroxides.

The term flake-form substrates is taken to mean all flake-form substrates that are known to the person skilled in the art. Suitable base substrates for the effect pigments, such as, for example, pearlescent pigments and interference pigments, are transparent or semitransparent flake-form substrates. Particularly suitable are phyllosilicates, talc, kaolin, flake-form iron oxides or aluminium oxides, glass flakes, SiO₂ flakes, TiO₂ flakes, flake-form mixed oxides, such as, for example, FeTiO₃, Fe₂TiO₅, or other comparable materials, depending on the respective legal approval for use in foods.

The size of the base substrates is not crucial per se and can be matched to the respective intended application. In general, the flake-form substrates have a thickness of 0.005 to 10 μm, in particular from 0.05 to 5 μm. The size in the two other dimensions is usually 1 to 500 μm, preferably 2 to 200 μm and in particular 5 to 150 μm. Very particularly preferred pearlescent pigments have particle sizes of 10-60 μm or 5-25 μm or 10-150 μm or 5-50 μm.

Preferred effect pigments, such as, for example, pearlescent pigments, are based on natural mica flakes, synthetic mica flakes, SiO₂ flakes, Al₂O₃ flakes, TiO₂ flakes, glass flakes, Fe₂O₃ flakes, in particular synthetic mica flakes, natural mica flakes and SiO₂ flakes (No. E555 or E551 in the list of food additives approved in the European Union). The synthetic flakes, such as, for example, synthetic mica flakes, SiO₂ flakes, Al₂O₃ flakes, TiO₂ flakes and glass flakes, may be doped or undoped. Suitable dopants are, inter alia, metal oxides, such as, for example, TiO₂, ZrO₂ and SnO₂.

The pearlescent pigments used are pigments based on flake-form, transparent or semitransparent substrates comprising, for example, phyllosilicates, such as, for example, natural mica, synthetic mica, talc, sericite or kaolin, or comprising glass or other silicate materials, which are coated with coloured or colourless metal oxides, such as, for example, TiO₂, titanium suboxides, titanium oxynitrides, Fe₂O₃, Fe₃O₄, SnO₂, ZnO and other metal oxides, alone or in a mixture in a homogeneous layer or in successive layers. Particularly preferred pearlescent pigments based on synthetic or natural mica flakes are coated with one or more metal oxides from the group TiO₂, Fe₂O₃ and Fe₃O₄ or mixtures thereof or have a multilayer coating consisting of alternating high- and low-refractive-index layers, such as, for example, TiO₂—SiO₂—TiO₂.

Preference is furthermore given to TiO₂— and/or Fe₂O₃-coated SiO₂ or Al₂O₃ flakes. The coating of the SiO₂ flakes with one or more metal oxides can be carried out, for example, as described in WO 93/08237 (wet-chemical coating) or DE-A 196 14 637 (CVD method).

The metal oxides applied to the flake-form substrates are also preferably materials in food grade which are suitable for human consumption (No. E171 (TiO₂) or E172 (iron oxide) in the list of food additives approved in the European Union).

Preference is furthermore given to pearlescent pigment mixtures which have different particle sizes. Particular effects can be achieved if pearlescent pigments having “small” particle sizes, such as, for example, 5-25 μm or 10-60 μm, are mixed with pearlescent pigments having “large” particle sizes, such as, for example, 10-150 μm.

The thickness of the individual layers, preferably one or more metal oxide layers, on the base substrate is preferably 10-500 nm, in particular 20-400 nm and very particularly preferably 30-350 nm.

In the case of multilayered pigments, which preferably have alternating high- and low-refractive-index layers (A)(B)(A) on the substrate surface, the high-refractive-index layer (layer A) generally has layer thicknesses of 10-500 nm, preferably 20-400 nm and in particular 30-350 nm. The thickness of the low-refractive-index layer (layer B) is preferably 10-500 nm, preferably 20-400 nm, in particular 30-350 nm.

High-refractive-index layers in this application are taken to mean layers having a refractive index of 1.8, such as, for example, TiO₂, Fe₂O₃ or Fe₃O₄, whereas low-refractive-index layers have a refractive index of <1.8, such as, for example, SiO₂, Al₂O₃ or AIO(OH).

The effect pigments, preferably pearlescent pigments, may comprise a plurality of identical or different combinations of layer packages, but coating of the substrate with only one layer package (A) (B) (A) in the case of multilayered pigments is preferred. In other to increase the tinting strength, the pigment according to the invention may comprise up to 4 layer packages, although in this case the thickness of all layers on the substrate should not exceed 3 μm. In the case of multilayered pigments having 3 or more layers on the substrate surface, an odd number of layers is preferably applied to the flake-form substrate, each with a high-refractive-index layer in the innermost and outermost position. A structure of three optical interference layers in the sequence (A) (B) (A) is particularly preferred. A suitable high-refractive-index layer is preferably TiO₂, Fe₂O₃ and/or Fe₃O₄ or a mixture of titanium oxide and iron oxide. The TiO₂ here can be in the rutile modification or in the anatase modification.

Suitable colourless low-refractive-index materials which are suitable for the coating (B) are preferably metal oxides or the corresponding oxide hydrates, such as, for example, SiO₂, Al₂O₃, AIO(OH), B₂O₃, MgF₂, MgSiO₃ or a mixture of the said metal oxides, in accordance with the legal approvals for use in foods or pharmaceutical products.

Particularly preferred pearlescent pigments for the foods sector are mica flakes (synthetic or natural) or SiO₂ flakes, which are covered with a metal oxide layer (TiO₂ or Fe₂O₃ or Fe₃O₄) having a thickness of 10 nm to 500 nm. The thicknesses of the flakes are in the range from 200 nm to 900 nm. Depending on the thickness of the flakes employed and the metal oxide layers applied and on the type of metal oxide, pigments of this type are distinguished by particularly intense interference colours and/or by strong angle-dependent colour flop effects. The latter are apparent inasmuch as an observer perceives different colours on changing his observation position relative to the pigmented object.

Preferred effect pigments are selected, in particular, from the pigments mentioned below:
natural mica flakes+TiO₂
natural mica flakes+Fe₂O₃
natural mica flakes+Fe₃O₄
natural mica flakes+TiO₂+Fe₂O₃
natural mica flakes+TiO₂+Fe₃O₄
natural mica flakes+Fe₂O₃+TiO₂
natural mica flakes+Fe₃O₄+TiO₂
natural mica flakes+TiO₂/Fe₂O₃ mixture
natural mica flakes+TiO₂/Fe₃O₄ mixture
synthetic mica flakes+TiO₂
synthetic mica flakes+Fe₂O₃
synthetic mica flakes+Fe₃O₄
synthetic mica flakes+TiO₂+Fe₂O₃
synthetic mica flakes+TiO₂+Fe₃O₄
synthetic mica flakes+Fe₂O₃+TiO₂
synthetic mica flakes+Fe₃O₄+TiO₂
synthetic mica flakes+TiO₂/Fe₂O₃ mixture
synthetic mica flakes+TiO₂/Fe₃O₄ mixture
SiO₂ flakes+TiO₂
SiO₂ flakes+Fe₂O₃
SiO₂ flakes+Fe₃O₄
SiO₂ flakes+TiO₂+Fe₂O₃
SiO₂ flakes+TiO₂+Fe₃O₄
SiO₂ flakes+Fe₂O₃+TiO₂
SiO₂ flakes+Fe₃O₄+TiO₂
SiO₂ flakes+TiO₂/Fe₂O₃ mixture
SiO₂ flakes+TiO₂/Fe₃O₄ mixture
natural mica flakes+TiO₂+SiO₂+TiO₂
natural mica flakes+Fe₂O₃+SiO₂+TiO₂
natural mica flakes+TiO₂+SiO₂+Fe₂O₃
natural mica flakes+TiO₂+SiO₂+Fe₃O₄
natural mica flakes+TiO₂/Fe₂O₃ mixture+SiO₂+Fe₂O₃
natural mica flakes+TiO₂/Fe₂O₃ mixture+SiO₂+TiO₂/Fe₂O₃ mixture
natural mica flakes+Fe₂O₃ mixture+SiO₂+TiO₂/Fe₂O₃ mixture
synthetic mica flakes+TiO₂+SiO₂+TiO₂
synthetic mica flakes+Fe₂O₃+SiO₂+TiO₂
synthetic mica flakes+TiO₂+SiO₂+Fe₂O₃
synthetic mica flakes+TiO₂+SiO₂+Fe₃O₄
synthetic mica flakes+TiO₂/Fe₂O₃ mixture+SiO₂+Fe₂O₃
synthetic mica flakes+TiO₂/Fe₂O₃ mixture+SiO₂+TiO₂/Fe₂O₃ mixture
synthetic mica flakes+Fe₂O₃ mixture+SiO₂+TiO₂/Fe₂O₃ mixture
SiO₂ flakes+TiO₂+SiO₂+TiO₂
SiO₂ flakes+Fe₂O₃+SiO₂+TiO₂
SiO₂ flakes+TiO₂+SiO₂+Fe₂O₃
SiO₂ flakes+TiO₂+SiO₂+Fe₃O₄
SiO₂ flakes+TiO₂/Fe₂O₃ mixture+SiO₂+Fe₂O₃
SiO₂ flakes+TiO₂/Fe₂O₃ mixture+SiO₂+TiO₂/Fe₂O₃ mixture
SiO₂ flakes+Fe₂O₃ mixture+SiO₂+TiO₂/Fe₂O₃ mixture.

The pearlescent pigment or mixture of pearlescent pigments is preferably employed in an amount of 0.05-45% by weight, in particular 0.5-25% by weight, particularly preferably 5-15% by weight, based on the total weight of the application medium.

Besides water and pearlescent pigment, the application medium preferably comprises a stabiliser (S), which serves to keep the effect pigment, preferably the pearlescent pigment, in suspension in the mixture, i.e. to prevent its sedimentation.

Stabilisers are known in a wide variety and are used as such. One group of possible stabilisers comprises a combination of colloidal microcrystalline cellulose (MCC) and carboxymethylcellulose (CMC). In particular, it is possible to employ the following stabilisers from the list of food additives approved in the European Union: E322, E401, E402, E403, E404, E405, E406, E407, E410, E412, E413, E414, E415, E416, E417, E418, E422, E423, E424, E425, E426, E440, E445, E460, E463, E464, E465, E466, E458, E469, E499, E551, E553, E554, E1201, E1202, E1404, E1410, E1412, E1413, E1414, E1420, E1422, E1440, E1442, E1450, E1520. In accordance with the invention, preference is given to E466 (sodium carboxymethylcellulose).

The stabiliser is preferably employed in an amount of 0.25-8% by weight, particularly preferably 0.5-4% by weight, very particularly preferably 1.0-3.0% by weight, based on the total weight of the application medium.

Furthermore, the application medium preferably comprises a moisture regulator (F), which effects rapid and uniform initial drying and final drying of the application medium on the surface.

Moisture regulators are known in a wide variety and are used as such. It is possible to employ the following moisture regulators, inter alia, from the list of food additives approved in the European Union: E420, E422, E964, E968, E1200, E1520. In accordance with the invention, preference is given to E1520 (1,2-propanediol).

The moisture regulator is preferably employed in an amount of 0.5-25% by weight, particularly preferably 2.0-15% by weight, very particularly preferably 3.0-11.0% by weight, based on the total weight of the application medium.

Furthermore, the application medium preferably comprises an emulsifier (E), which ensures homogeneous mixing of the components present in the application medium.

Emulsifiers are known in a wide variety and are used as such. It is possible to employ, inter alia, the following emulsifiers from the list of food additives approved in the European Union: E322, E405, E426, E432, E442, E444, E459, E470a, E471, E472b, E472e, E473, E476, E481, E483, E491, E920. In accordance with the invention, preference is given to E322 (soya or sunflower lecithin).

The emulsifier is preferably employed in an amount of 0.25-5% by weight, particularly preferably 0.5-3% by weight, very particularly preferably 1.0-2.0% by weight, based on the total weight of the application medium.

Furthermore, the application medium preferably comprises a preservative (K), which ensures microbiological stability of the application medium mixture.

Preservatives are known in a wide variety and are used as such. It is possible to employ, inter alia, preservatives from the list of food additives approved in the European Union: E200, E202, E203, E210, E211, E212, E213, E214, E215, E218, E219, E220, E221, E222, E223, E224, E226, E228, E231, E232, E234, E235, E239, E242, E249, E250, E251, E252, E260, E261, E262, E263, E280, E281, E282, E283, E284, E285, E1105, E1519. Preferred preservatives in accordance with the invention are combinations of E202 with food acids, such as, for example, E330, E270 or other food acids.

The preservative mixture is preferably employed in an amount of 0.1-1.5% by weight, preferably 0.2-1% by weight, particularly preferably 0.25-0.5% by weight, based on the total weight of the application medium. The preservative mixture typically consists of 75-90% by weight of food acid and 10-25% by weight of the actual preservative.

Further components (W), such as, for example, sweeteners, flavourings or other dyes, may optionally be added to the application medium mixture.

In a preferred embodiment, the application medium comprises water+S+F+E+K+effect pigment, preferably at least one pearlescent pigment, + optionally W.

In a further embodiment of the invention, the application medium mixture may be diluted further with water, or some of the water in the basic recipe may already have been replaced by ethanol. The latter procedure shortens the drying time of the applied application medium and increases the microbiological stability of the application medium mixture.

The application medium mixture can be prepared using conventional mixing methods and stirrers, typically using an open vessel. Components S, F, E, K, in each case if present, and optionally W of the application medium mixture are introduced into the water-charged vessel in any desired sequence or all simultaneously and subsequently mixed by stirring. The mixing process can optionally be accelerated by warming.

The pearlescent pigments individually, combinations of pearlescent pigments, or combinations of the two above-mentioned options with further dyes/pigments, such as, for example, beetroot, β-carotene, paprika E160c, anthocyanins, caramel, biochar, carmine E120, riboflavin, caramel colouring E150, curcumin, etc., mineral food dyes (iron oxide, titanium dioxide, etc.) or synthetic food dyes (E131, E129, E133, E127, E141, E102, E122, E124, E110, E123, E132, E104, etc.), are then added to the prepared water-based mixture of components S, F, E, K and optionally W with further stirring. The same also applies to the use in combination with so-called colouring plants and fruit extracts, colouring foods (such as, for example, black carrot, stinging nettle, spirulina, etc.). Flavourings of any type and sweeteners, such as, for example, aspartame, acesulfame K, stevia, saccharin, cyclamate, etc., can be added to the medium if desired.

The application medium can be applied to surfaces, in particular surfaces of foods, by means of simple methods, for example by manual application. The application medium can also be used on surfaces as decoration without prior coating after corresponding drying. For this purpose, it is converted into the desired shape in advance by means of corresponding moulds or underlays.

The application medium according to the invention, which is simple to prepare and consists exclusively of components which are approved under food law, can be used for the colouring of surfaces, in particular of a multiplicity of very different foods.

Pearlescent pigments may be present in the application medium in a broad concentration range from 0.05-45% by weight. The present invention makes novel and more intense pearlescent effects possible. Products which could hitherto be coloured with pearlescent pigments only with difficulty or without a satisfactory effect can be coloured by means of the present invention. No sedimentation of the pearlescent pigments occurs after incorporation into the application medium. Pearlescent pigments can be combined with other dyes/pigments (such as, for example, naturally colouring plant extracts, synthetic dyes). Individual addition of sweeteners and flavourings of any type is also possible. The application medium can be applied to the products by means of simple methods, such as, for example, by manual application. The avoidance of pearlescent dust during the colouring process prevents contamination from occurring.

The list of food additives approved in the European Union is incorporated herein by reference. The identity of each of the various additives identified by reference numbers such as, e.g., E200, is publicly available, for example, from the internet or the European Union and is also well-known by a person skilled in the art.

The following examples are intended to explain the invention, but without limiting it. Unless indicated otherwise, all percentages in this application are percent by weight.

### EXAMPLES

### Example 1: Colouring of Hard-Boiled Eggs

### Application Medium:

Application medium:

| | |
|---|---|
| 80% by weight of water | |
| 5.75% by weight of 1,2-propanediol E1520 | Merck KGaA |
| 2.4% by weight of sodium carboxymethylcellulose | Roeper |
| 1.5% by weight of soya or sunflower lecithin | Sternchemie |
| 0.3% by weight of citric acid (crystalline) | Merck KGaA |
| 0.05% by weight of potassium sorbate | Merck KGaA |
| 10% by weight of pearlescent pigment (A1-A11) | Merck KGaA |

| | |
|---|---|
| A1) | Candurin ® Gold Lustre (mica + titanium dioxide + iron oxide) |
| A2) | Candurin ® Brown Amber (mica + iron oxide) |
| A3) | Candurin ® Gold Shimmer (mica + titanium dioxide) |
| A4) | Candurin ® Silver Lustre (mica + titanium dioxide) |
| A5) | Candurin ® Silver Lustre + Candurin ® Gold Lustre |
| | (9:1% by weight) |
| A6) | Candurin ® Silver Lustre + E131 (weight ratio: 0.005:0.5) |
| A7) | Candurin ® Silver Lustre + E153 or E172 black |
| | (weight ratio: 0.01:1) |
| A8) | Candurin ® NXT Ruby Red: (silicon dioxide + iron oxide) |
| A9) | Candurin ® Red Lustre (mica + iron oxide) |
| A10) | Candurin ® Silver Lustre + Candurin ® Red Lustre |
| | (weight ratio: 8:2) |
| A11) | Candurin ® Silver Lustre + Candurin ® Silver Sparkle |
| | (weight ratio: 8:2), both mica + titanium dioxide: Silver Sparkle |
| | with a greater particle diameter |

### Preparation of the Application Medium

Water is initially introduced with potassium sorbate and citric acid, stirred using a paddle stirrer, and the mixture is heated to a maximum of 80° C. During the heating, sodium carboxymethylcellulose is added with constant stirring. Stirring is continued (about 45 min.) until all the cellulose has dissolved. Alternatively, the mixture can be stirred without supply of heat for 3 h.

As soon as all the sodium carboxymethylcellulose has dissolved in the water, 1,2-propanediol and lecithin are added at room temperature, and stirring is continued until all the ingredients are uniformly distributed. The amount of pigment indicated is then stirred in.

### Colouring

Eggs are hard-boiled and then coloured by means of an application medium according to the invention. To this end, a small amount of the medium is placed on a glove. The other hand also wears a glove. The boiled egg should be warm and dry. The application medium is distributed uniformly on the warm egg shell by rubbing to and from in the hands. Due to the egg's residual heat, the application medium dries quickly, giving a very uniform pearlescent colouration. The eggs can have white or brown shells, depending on the pearlescent pigment selected.

Different pearlescent pigment amounts of 2-17% by weight result in different intensities of the resultant pearlescent effect.

There is no colour penetration through the egg shell.

### Example 2: Chocolate and Pralines

### Example 2.1: Ready-Made Pralines, Chocolates and Chocolate-Coated Products in General can be Embellished or Decorated Subsequently with the Pearlescent Pigment Application Medium

### Application Medium:

Application medium:

| | |
|---|---|
| 60% by weight of water | |
| 20% by weight of ethanol (96% by vol.) | Merck KGaA |
| 5.75% by weight of 1,2-propanediol E1520 | Merck KGaA |
| 2.4% by weight of sodium carboxymethylcellulose | Roeper |
| 1.5% by weight of soya or sunflower lecithin | Sternchemie |
| 0.3% by weight of citric acid (crystalline) | Merck KGaA |
| 0.05% by weight of potassium sorbate | Merck KGaA |
| 10% by weight of pearlescent pigment (selected from | Merck KGaA |
| A1)-A11) in accordance with Example 1) + | |
| optionally sweetener and/or flavourings as desired | |

### Preparation of the Application Medium as in Example 1

### Colouring:

The application medium is applied to the areas of the chocolate product to be coloured by means of a fine nozzle, spatula or brush. The colour coating is then dried at room temperature until completely dry.

The viscosity of the medium can be reduced by additional addition of water. This enables the medium, where appropriate, to be applied better to certain product areas. The drying time is consequently increased.

### Example 2.2: Pre-Colouring of Chocolate Moulds

### Application Medium:

Application medium:

| | |
|---|---|
| 80% by weight of water | |
| 10.75% by weight of 1,2 propanediol E1520 | Merck KGaA |
| 2.4% by weight of sodium carboxymethylcellulose | Roeper |
| 1.5% by weight of soya or sunflower lecithin | Sternchemie |
| 0.3% by weight of citric acid (crystalline) | Merck KGaA |
| 0.05% by weight of potassium sorbate | Merck KGaA |
| + pearlescent pigment (selected from | Merck KGaA |
| A1)-A11) in accordance with Example 1) | |

### Preparation of the Application Medium:

The components are mixed as described in Example 1. Before addition of the pigment, a further 50% by weight of water are added and stirring is continued until everything is homogeneously distributed.

5% by weight of the selected pearlescent pigment in accordance with A1) to A11) are added to this mixture.

### Colouring:

Chocolate moulds made from plastic (for example polycarbonate) or metal can be pre-decorated very simply using the application medium according to the invention. In this case, the pigment-containing application medium is introduced into the mould at the desired points. The mould is subsequently dried in a fan-assisted oven or drying cabinet. The colouring of the mould can be carried out by suitable nozzles, pipettes, brushes or by hand.

The viscosity of the medium can be reduced by additional addition of water. This enables the medium, where appropriate, to be applied better to certain product areas. The drying time is consequently increased.

Some of the water can optionally be replaced by ethanol (food grade 80-99.99% by vol.) during preparation of the application medium. This accelerates final drying. 5-30% of the water can be replaced by ethanol.

### Example 2.3: Furthermore, it is Possible to Colour Chocolate Products UniFormly by Hand Analogously to Example 1

### Example 3: Decoration of Other Confectionery

Other confectionery, such as, for example, marzipan or fondant products, can be decorated or embellished subsequently with the application medium according to the invention analogously to the decoration of chocolate and pralines (Example 2).

In contrast to chocolate and pralines, these products can be dried at higher temperatures after the application.

Otherwise, the same recipe variations, preparation processes and colouring possibilities as described in Example 2 apply.

### Example 4: Decoration, Colouring of Cereals

Extruded breakfast cereals can only be coloured with difficulty using the conventional pearlescence application methods. Application by means of a diluted sugar solution still gives the best results, but this also increases the proportion of sugar in the product. In addition, crystallising sugar can lead to a reduction in the pearlescent effect.

Cereals can also be coloured without difficulty by means of the pearlescence-containing application medium developed.

### Application Medium (Base):

Application medium (base):

| | |
|---|---|
| 70% by weight of water | |
| 23% by weight of ethanol (96% by vol.) | Merck KGaA |
| 3.5% by weight of 1,2 propanediol E1520 | Merck KGaA |
| 2.0% by weight of sodium carboxymethylcellulose | Roeper |
| 1.2% by weight of soya or sunflower lecithin | Sternchemie |
| 0.25% by weight of citric acid (crystalline) | Merck KGaA |
| 0.05% by weight of potassium sorbate | Merck KGaA |

Optionally sweeteners and flavourings as desired

The corresponding pearlescent pigments, individually or in combination, and/or additionally other dyes or colouring fruit and plant extracts, depending on the pearlescent effect desired, are then added to this base prepared as in Example 1.

### Colouring:

### Example 4.1: Chocolate Cereals, Round, Extruded

Cereals are introduced into a cooker. This is fitted with lifter blades. Drying air is switched on (40-50° C.)

| | |
|---|---|
| Initial weight of cereals: | 40 g |
| Application medium: | |
| 10% by weight of Candurin ® Silver Lustre | Merck KGaA |
| 0.05% by weight of biochar | CHR Hansen |
| 89.95% by weight of application medium base | |
| (preparation as above) | |

A total of 60 g (=15% of the amount of cereals) of application medium is applied little by little to the cereals rotating in the cooker. 10 g of the application medium are applied per application. This is distributed on the cereals. Drying air is directed onto the cereals. As soon as an application has dried, a corresponding application amount can be re-applied to the cereals, until the desired colour effect has been achieved. The cereals are then dried, for example in a drying cabinet or oven, until the initial moisture content has been restored.

### Example 4.2: Chocolate Cereals, Round, Extruded

The procedure is as in Example 4.1.

| | |
|---|---|
| Initial weight of cereals: | 400 g |
| Application medium: | |
| 5% by weight of Candurin ® Gold Lustre | Merck KGaA |
| 3% by weight of Candurin ® Gold Sparkle | Merck KGaA |
| 92% by weight of application medium base (preparation | |
| as above) | |
| Amount applied: | 50 g (=12.5%) |

### Example 4.3: Grain Cereals, Oblong, Extruded

The procedure is as in Example 4.1.

| | |
|---|---|
| Initial weight of cereals: | 500 g |
| Application medium: | |
| 12% of Candurin ® NXT Ruby Red | Merck KGaA |
| 88% of application medium base (preparation | |
| as above) | |
| Amount applied: | 40 g (=8%) |

### Example 5: Colouring and Decoration of Popcorn

Colouring of popcorn via the sugar or fat used for popcorn production comes into consideration as a possible (conventional) option. Rapid caramelisation of the sugar would, however, lead to masking of the pearlescent effect. It would usually be possible to colour salty popcorn via the fat used for popcorn production. Colouring via fat also works only satisfactorily in the case of sealed microwave popcorn, but the use amounts of pigment necessary are very large.

Analogously to cereals, popcorn can easily be coloured or decorated by means of the transparent application medium according to the invention comprising pearlescent pigments.

### Example 5.1: Sugared Popcorn

| | |
|---|---|
| Initial weight of popcorn: | 300 g |
| Application medium: | |
| 5% by weight of Candurin ® Red Lustre | Merck KGaA |
| 3% by weight of Candurin ® Red Sparkle | Merck KGaA |
| 92% by weight of application medium base | |
| (composition and preparation as in Example 4) | |
| Amount applied: | 50 g (=16.67%) |

A total of 50 g (=16.67%) of application medium is applied little by little to the popcorn rotating in a cooker. 10-15 g of the medium are applied per application. This is distributed on the popcorn while drying air is fed in. As soon as an application has dried, a further application amount can be applied to the popcorn, until the desired colour effect has been achieved. The popcorn is then dried, for example in a drying cabinet or oven, until the initial moisture content has been restored.

### Example 5.2: Sugared Popcorn

| | |
|---|---|
| Initial weight of popcorn: | 300 g |
| Application medium: | |
| 10% by weight of Candurin ® Brown Amber | Merck KGaA |
| 90% by weight of application medium base | |
| (composition and preparation as in Example 4) | |
| Amount applied: | 60 g (=20%) |

### Example 6: Dragees (Chocolate, Sugar/Sugar-Free)

The pearlescent pigment-containing application medium is applied manually to the chocolate dragees rotating in the cooker analogously to conventional sugar coating. Here too, layers are applied successively until the desired effect has been achieved. As soon as a layer has dried, further addition can take place. Application of a small amount gives a marble effect, while application of an increased amount leads to a very uniform pearlescent colouration.

Application medium (base):

| | |
|---|---|
| 70% by weight of water | |
| 23% by weight of ethanol (96% by vol.) | Merck KGaA |
| 3.5% by weight of 1,2-propanediol E1520 | Merck KGaA |
| 2.0% by weight of sodium carboxymethylcellulose | Roeper |
| 1.2% by weight of soya or sunflower lecithin | Sternchemie |
| 0.25% by weight of citric acid (crystalline) | Merck KGaA |
| 0.05% by weight of potassium sorbate | Merck KGaA |

Optional addition of sweeteners and flavourings. The ethanol content can be varied in order to adapt the recipe or even replaced entirely by water and 1,2-propanediol.

### Example 6.1: Hazelnuts Coated with Dark Chocolate

Dragees are introduced into a cooker. This is fitted with lifting blades. Drying air is switched on (25° C.)

| | | |
|---|---|---|
| Initial weight | 1000 g | |
| of dragees: | | |
| Application | 10% by weight of Candurin ® Silver Lustre | Merck KGaA |
| medium: | 90% by weight of application medium base | |
| | (composition as in Example 6, preparation | |
| | as in Example 4) | |
| Amount | 80 g (=8%) | |
| applied: | | |

### Example 6.2: Chocolate Coffee Beans Coated with Milk Chocolate

### Preparation as in Example 6.1

| | | |
|---|---|---|
| Initial weight | 1200 g | |
| of dragees: | | |
| Application | 10% by weight of Candurin ® Brown Amber | Merck KGaA |
| medium: | 90% by weight of application medium base | |
| | (composition as in Example 6, preparation | |
| | as in Example 4) | |
| Amount | 95 g (=7.9%) | |
| applied: | | |

### Example 6.3: Raisins Coated with White Chocolate

### Preparation as in Example 6.1

| | | |
|---|---|---|
| Initial weight | 1000 g | |
| of dragees: | | |
| Application | 8% by weight of Candurin ® Gold Shimmer | Merck KGaA |
| medium: | 92% by weight of application medium base | |
| | (composition as in Example 6, preparation | |
| | as in Example 4) | |
| Amount | 60 g (=6%) | |
| applied: | | |

### Example 6.4: Sugar-Coated Chocolate Buttons, White

| | | |
|---|---|---|
| Initial weight | 1000 g | |
| of dragees: | | |
| Application | 8% by weight of Candurin ® Brown Amber | Merck KGaA |
| medium: | 92% by weight of application medium base | |
| | (composition as in Example 6, preparation | |
| | as in Example 4) | |
| Amount | 100 g (=10%) | |
| applied: | | |

### Example 6.5: Isomalt-Coated Chewing Gum Pellets, White

| | | |
|---|---|---|
| Initial weight | 1000 g | |
| of dragees: | | |
| Application | 8% by weight of Candurin ® Brown Amber | Merck KGaA |
| medium: | 92% by weight of application medium base | |
| | (composition as in Example 6, preparation | |
| | as in Example 4) | |
| Amount | 80 g (=8%) | |
| applied: | | |

### Example 6.6: Sugar-Coated Almonds, Red

| | | |
|---|---|---|
| Initial weight | 800 g | |
| of dragees: | | |
| Application | 8% by weight of Candurin ® Silver Lustre | Merck KGaA |
| medium: | 92% by weight of application medium base | |
| | (composition as in Example 6, preparation | |
| | as in Example 4) | |
| Amount | 72 g (=9%) | |
| applied: | | |

### Example 6.7: Further Surface Optimisation of Dragees

Both in the case of the chocolate-coated products (Examples 6.1-6.3), and in the case of the sugar- or sugar-free-coated products (Examples 6.4-6.6), the pearlescence can be improved further if a mixture of shellac, ethanol and pearlescent pigment is also applied after application of the application medium and after a certain drying time. Ideally, this can be the pearlescent pigment(s) already present in the medium, but other combinations are also possible. The shellac/ethanol/pearlescent pigment mixture is spread manually over the dragees rotating in the cooker and dried using a stream of air. Alternatively, the mixture can also be sprayed.

### Composition (Example):

90% by weight of ethanolic shellac solution (2-50% shellac content) (for example Capol® 425 or Crystallac® (Mantrose-Haeuser Co., Inc.)
10% by weight of pearlescent pigment (selected from A1-A11 in accordance with Example 1)

Before addition of the pearlescent pigment, the shellac solution can be diluted further with ethanol (for example 1 part of shellac solution diluted with twice or 5 times the amount of ethanol (food grade).

Amount applied: 5-40 g/kg of product—depending on the dilution
Amount of pearlescent pigment in the shellac solution (2-50% shellac content)+1-40% of pigment, preferably: 5-25% of pigment

### Example 7: Colouring of Compressed Products

Direct colouring of compressed products with pearlescent pigments makes little sense since firstly the resultant pearlescence is not very visible, and secondly high pigment use amounts are necessary. Spraying-on usually requires long production times and often also leads to damage to the compressed products during the colouring process.

Uniform colouring of the compressed products can be achieved by means of the application medium according to the invention. The colouring is advantageously carried out in a coating cooker and follows the principle described in Example 6. A further advantage of the application medium according to the invention consists in that it rapidly forms a protective layer around the compressed products. This greatly reduces the “rounding” which is often observed. An interesting “marble pearlescent effect” can rapidly be achieved by means of only a few applications.

If desired, the compressed products can be dried to the original moisture content of the product in an oven, etc., after the colouring.

Application medium (base):

| | |
|---|---|
| 72% by weight of water | |
| 20% by weight of ethanol (96% by vol.) | Merck KGaA |
| 4.5% by weight of 1,2-propanediol E1520 | Merck KGaA |
| 2.0% by weight of sodium carboxymethylcellulose | Roeper |
| 1.2% by weight of soya or sunflower lecithin | Sternchemie |
| 0.25% by weight of citric acid (crystalline) | Merck KGaA |
| 0.05% by weight of potassium sorbate | Merck KGaA |

In order to adapt the recipe, sweeteners and/or flavourings can optionally be added. The ethanol content can be varied in order to adapt the recipe, or ethanol can even be replaced entirely by water and 1,2-propanediol.

### Example 7.1: Peppermint Compressed Products, White

| | | |
|---|---|---|
| Initial weight | 500 g | |
| of product: | | |
| Pearlescent | 8% by weight of Candurin ® Silver Sparkle, | Merck |
| medium: | 92% by weight of application medium | KGaA |
| | (composition as in Example 7, preparation as in | |
| | Example 4) | |
| Amount | 40 g (=8%) | |
| applied: | | |

### Example 7.2: Lemon Compressed Products, Pale Yellow

| | | |
|---|---|---|
| Initial weight | 500 g | |
| of product: | | |
| Pearlescent | 6% by weight of Candurin ® Gold Lustre Sparkle, | Merck |
| medium: | 94% by weight of application medium base | KGaA |
| | (composition as in Example 7, preparation as in | |
| | Example 4) | |
| Amount | 50 g (=10%) | |
| applied: | | |

### Example 8: Colouring and Decoration of Marshmallows

In the colouring of marshmallows with pearlescent pigments, satisfactory results have not been achieved to date. The application of the application medium according to the invention enables pearlescent effects to be achieved easily on the surface of marshmallows.

The application is carried out in a coating cooker. The process sequence is carried out as described in Example 6.

The composition of the medium is analogous to that of compressed products (Example 7). The same applies to possible modifications thereof.

### Example 8.1: Colouring of Marshmallows, White

| | | |
|---|---|---|
| Initial weight | 300 g | |
| of product: | | |
| Pearlescent | 10% by weight of Candurin ® Silver Sparkle | Merck |
| medium: | 90% by weight of application medium base | KGaA |
| | (composition as in Example 7, preparation as in | |
| | Example 4) | |
| Amount | 51 g (=17%) | |
| applied: | | |

### Example 8.2: Colouring of Marshmallows, Pale Green

| | | |
|---|---|---|
| Initial weight | 300 g | |
| of product: | | |
| Pearlescent | 10% by weight of Candurin ® Gold Sparkle | Merck |
| medium: | 90% by weight of application medium base | KGaA |
| | (composition as in Example 7, preparation | |
| | as in Example 4) | |
| Amount | 51 g (=17%) | |
| applied: | | |

### Example 8.3: Colouring of Marshmallows, Pink

| | | |
|---|---|---|
| Initial weight | 300 g | |
| of product: | | |
| Pearlescent | 8% by weight of Candurin ® Silver Sparkle | Merck |
| medium: | 92% by weight of base (composition as in | KGaA |
| | Example 7, preparation as in Example 4) | |
| Amount | 42 g (=14%) | |
| applied: | | |

### Example 9: Production of Decoration Lace and Decoration Sheets

Decoration lace is taken to mean filigree shapes and decorations which are produced by thinly spreading the application medium according to the invention in silicone moulds. These are removed from the moulds after a certain drying time and can be used for the embellishment of, for example, confectionery, ice cream, chocolate, eggs, pralines, baked goods, cakes and gateaux, deserts, pates, aspics and other foods. The drying time here can be set so that the products still exhibit a certain elasticity.

Use of the resultant decorations as edible body jewellery is also possible. Here, the decorations are applied to the corresponding body areas by means of suitable adhesives. The paste when used as edible cosmetic can furthermore also be applied directly to the body areas to be coloured. The body heat causes rapid drying on the skin.

Decoration sheets are taken to mean very thin, sheet-form structures which are produced by spreading out the pearlescent medium very thinly on flat substrates. These substrates may consist, for example, of plastic or metal. After the spreading out, the products are dried, for example in a drying cabinet or oven, until completely dry. After drying, the decoration sheets can be removed from the substrate and can then be used as a whole, in part or correspondingly cut or chopped into small pieces for the embelliushment of foods, as correspondingly listed, for example, in the case of the decoration lace described above.

### Example 9.1: Recipe for Decoration Lace

| | |
|---|---|
| 76% by weight of water | |
| 9.75% by weight of 1,2-propanediol E1520 | Merck KGaA |
| 2.4% by weight of sodium carboxymethylcellulose | Roeper |
| 1.5% by weight of soya or sunflower lecithin | Sternchemie |
| 0.3% by weight of citric acid (crystalline) | Merck KGaA |
| 0.05% by weight of potassium sorbate | Merck KGaA |
| 10% by weight of pearlescent pigment (selected from | Merck KGaA |
| A1) to A11) in accordance with Example 1) | |

In order to adapt the recipe, sweeteners and/or flavourings can optionally be added. The proportion of water and 1,2-propanediol can be varied in order to adapt the recipe specifically to the customer.

Ethanol can likewise be added in order to accelerate the drying.

After preparation, the application medium is spread thinly in silicone moulds and dried at about 70° C. in a drying cabinet for 2-3 hours. The decorations can then carefully be removed from the mould.

Colouring possibilities of the application medium with pearlescent pigments, combinations of pearlescent pigments or combinations of pearlescent pigments with other natural, mineral and synthetic food dyes or pigments exist, as indicated, for example, in Example 1.

### Example 9.2: Recipe for Decoration Sheets

| | |
|---|---|
| 80% by weight of water | |
| 5.75% by weight of 1,2 propanediol E1520 | Merck KGaA |
| 2.4% by weight of sodium carboxymethylcellulose | Roeper |
| 1.5% by weight of soya or sunflower lecithin | Sternchemie |
| 0.3% by weight of citric acid (crystalline) | Merck KGaA |
| 0.05% by weight of potassium sorbate | Merck KGaA |
| 10% by weight of pearlescent pigment (selected from | Merck KGaA |
| A1) to A11) in accordance with Example 1) | |

In order to adapt the recipe, sweeteners and/or flavourings can optionally be added. The proportion of water and 1,2-propanediol can be varied in order to adapt the recipe specifically to the customer.

Ethanol can likewise be added in order to accelerate the drying.

After production, the application medium is spread out very thinly on suitable substrates (plastic, metal, etc.) and subsequently dried in a drying cabinet or oven at about 70° C. until completely dry. The decoration sheets can then be detached completely and used as a whole, in part or cut into small pieces as decoration or embellishments for food products, as described under decoration lace.

The pearlescent and interference pigments used in the examples (Candurin® pigments from Merck) based on mica or SiO₂ flakes have the following composition and particle sizes:

| | |
|---|---|
| Candurin ® Gold Lustre: | mica (E555) coated with TiO₂ (E171) and |
| | Fe₂O₃ (E172ii); 10-60 μm |
| Candurin ® Brown Amber: | mica (E555) coated with Fe₂O₃ (E172ii); |
| | 10-60 μm |
| Candurin ® Gold Shimmer: | mica (E555) coated with TiO₂ (E171); |
| | 10-60 μm |
| Candurin ® Silver Lustre: | mica (E555) coated with TiO₂ (E171); |
| | 10-60 μm |
| Candurin ® Silver Sparkle: | mica (E555) coated with TiO₂ (E171); |
| | 10-150 μm |
| Candurin ® Red Sparkle: | mica (E555) coated with Fe₂O₃ (E172ii); |
| | 10-150 μm |
| Candurin ® NXT Ruby Red: | SiO₂ flakes (E551) coated with Fe₂O₃ |
| | (E172ii); 5-50 μm |
| Candurin ® Red Lustre: | mica (E555) coated with Fe₂O₃ (E172ii); |
| | 10-60 μm |

The preceding examples can be repeated with similar success by substituting the generically or specifically described reactants and/or operating conditions of this invention for those used in the preceding examples.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

From the foregoing description, one skilled in the art can easily ascertain the essential characteristics of this invention and, without departing from the spirit and scope thereof, can make various changes and modifications of the invention to adapt it to various usages and conditions.

The entire disclosure[s] of all applications, patents and publications, cited herein and of corresponding German application No. DE 102017001107.9, filed Feb. 7, 2017, are incorporated by reference herein.

1. A process for coloring a surface with effect pigments based on a flake-form substrate, comprising applying to said surface the effect pigments that have been incorporated into a pasty, transparent, water-based material that forms an application medium.

2. The process according to claim 1, wherein the surface is a food surface or the surface of the human body.

3. The process according to claim 1, wherein the effect pigments are pearlescent pigments, interference pigments or multilayered pigments.

4. The process according to claim 1, wherein the flake-form substrate is a natural mica flake, a synthetic mica flake, talc, kaolin, a glass flake, a silicon dioxide flake, a titanium dioxide flake, an aluminium oxide flake or an iron oxide flake.

5. The process according to claim 1, wherein the flake-form substrate is completely coated with one or more layers of metal oxides and/or metal oxide mixtures.

6. The process according to claim 1, wherein the flake-form substrate is coated with titanium dioxide and/or iron oxide.

7. The process according to claim 1, wherein the effect pigments are one of the following
natural mica flakes+TiO₂
natural mica flakes+Fe₂O₃
natural mica flakes+Fe₃O₄
natural mica flakes+TiO₂+Fe₂O₃
natural mica flakes+TiO₂+Fe₃O₄
natural mica flakes+Fe₂O₃+TiO₂
natural mica flakes+Fe₃O₄+TiO₂
natural mica flakes+TiO₂/Fe₂O₃ mixture
natural mica flakes+TiO₂/Fe₃O₄ mixture
synthetic mica flakes+TiO₂
synthetic mica flakes+Fe₂O₃
synthetic mica flakes+Fe₃O₄
synthetic mica flakes+TiO₂+Fe₂O₃
synthetic mica flakes+TiO₂+Fe₃O₄
synthetic mica flakes+Fe₂O₃+TiO₂
synthetic mica flakes+Fe₃O₄+TiO₂
synthetic mica flakes+TiO₂/Fe₂O₃ mixture
synthetic mica flakes+TiO₂/Fe₃O₄ mixture
SiO₂ flakes+TiO₂
SiO₂ flakes+Fe₂O₃
SiO₂ flakes+Fe₃O₄
SiO₂ flakes+TiO₂+Fe₂O₃
SiO₂ flakes+TiO₂+Fe₃O₄
SiO₂ flakes+Fe₂O₃+TiO₂
SiO₂ flakes+Fe₃O₄+TiO₂
SiO₂ flakes+TiO₂/Fe₂O₃ mixture
SiO₂ flakes+TiO₂/Fe₃O₄ mixture
natural mica flakes+TiO₂+SiO₂+TiO₂
natural mica flakes+Fe₂O₃+SiO₂+TiO₂
natural mica flakes+TiO₂+SiO₂+Fe₂O₃
natural mica flakes+TiO₂+SiO₂+Fe₃O₄
natural mica flakes+TiO₂/Fe₂O₃ mixture+SiO₂+Fe₂O₃
natural mica flakes+TiO₂/Fe₂O₃ mixture+SiO₂+TiO₂/Fe₂O₃ mixture
natural mica flakes+Fe₂O₃ mixture+SiO₂+TiO₂/Fe₂O₃ mixture
synthetic mica flakes+TiO₂+SiO₂+TiO₂
synthetic mica flakes+Fe₂O₃+SiO₂+TiO₂
synthetic mica flakes+TiO₂+SiO₂+Fe₂O₃
synthetic mica flakes+TiO₂+SiO₂+Fe₃O₄
synthetic mica flakes+TiO₂/Fe₂O₃ mixture+SiO₂+Fe₂O₃
synthetic mica flakes+TiO₂/Fe₂O₃ mixture+SiO₂+TiO₂/Fe₂O₃ mixture
synthetic mica flakes+Fe₂O₃ mixture+SiO₂+TiO₂/Fe₂O₃ mixture
SiO₂ flakess+TiO₂+SiO₂+TiO₂
SiO₂ flakess+Fe₂O₃+SiO₂+TiO₂
SiO₂ flakess+TiO₂+SiO₂+Fe₂O₃
SiO₂ flakess+TiO₂+SiO₂+Fe₃O₄
SiO₂ flakess+TiO₂/Fe₂O₃ mixture+SiO₂+Fe₂O₃
SiO₂ flakess+TiO₂/Fe₂O₃ mixture+SiO₂+TiO₂/Fe₂O₃ mixture or
SiO₂ flakess+Fe₂O₃ mixture+SiO₂+TiO₂/Fe₂O₃ mixture.

8. The process according to claim 1, wherein the application medium contains 0.05-45% by weight pearlescent pigment, based on the total weight of the application medium.

9. The process according to claim 1, wherein the application medium contains (i) one type of effect pigment, (ii) more than one type of effect pigments (iii) one type of effect pigment and one or more dyes and/or further pigments or (iv) more than one type of effect pigments and one or more dyes and/or further pigments.

10. The process according to claim 1, wherein the application medimum further contains one or more flavourings and/or sweeteners.

11. The process according to claim 1, wherein the pasty, transparent water-based material additionally comprises at least one component selected from the group consisting of stabilisers, moisture regulators, emulsifiers and preservatives.

12. The process according to claim 1, wherein the pasty, transparent water-based material comprises water, at least one stabiliser, at least one moisture regulator, at least one emulsifier and at least one preservative and optionally further components.

13. The process according to claim 1, wherein the application medium contains the stabiliser in an amount of 0.25-8% by weight, based on the total weight of the application medium.

14. The process according to claim 1, wherein the application medium contains the moisture regulator in an amount of 0.5-25% by weight, based on the total weight of the application medium.

15. The process according to claim 1, wherein the application medium contains the emulsifier in an amount of 0.25-5% by weight, based on the total weight of the application medium.

16. The process according to claim 1, wherein the application medium contains the preservative in an amount of 0.1-1.5% by weight, based on the total weight of the application medium.

17. The process according to claim 1, wherein the application medium additionally comprises ethanol.

18. The process according to claim 1, comprising converting the application medium firstly into a predetermined shape by a mould or underlay and then applying the resultant shaped application medium to the surface.

19. A material comprising water, at least one stabiliser, at least one moisture regulator, at least one emulsifier, at least one preservative and optionally further components.

20. An application medium, suitable for the application of effect pigments based on platelet-shaped substrates to a surface, comprising one or more effect pigments and the material according to claim 19.

21. A surface that has been provided with the medium according to claim 20.

22. A surface according to claim 21, which is the surface of a food or the surface of the skin of the human body.
